(19) United States
(12) Patent Application Publication
(10) Pub. No.: US 2017/0258302 A1
(43) Pub. Date: Sep. 14, 2017

(54) ENDOSCOPE AND ENDOSCOPE SYSTEM

(71) Applicant: OLYMPUS CORPORATION, Tokyo (JP)

(72) Inventors: Takeshi TAKAHASHI, Tokyo (JP); Yasuhito KURA, Tokyo (JP); Kazuki HONDA, Tokyo (JP); Kazuhiko HINO, Tokyo (JP); Kento HASHIMOTO, Tokyo (JP); Takeo SUZUKI, Tokyo (JP)

(73) Assignee: OLYMPUS CORPORATION, Tokyo (JP)

(21) Appl. No.: 15/606,363

(22) Filed: May 26, 2017

Related U.S. Application Data
(63) Continuation of application No. PCT/JP2015/079677, filed on Oct. 21, 2015.

(30) Foreign Application Priority Data
Nov. 27, 2014 (JP) 2014-240324

(51) Int. Cl. A61B 1/00; A61B 1/05

(52) U.S. Cl. CPC A61B 1/00181; A61B 1/00096; A61B 1/05; A61B 1/00174; A61B 1/0661

(57) ABSTRACT

An endoscope includes: a first image acquisition portion configured to acquire a first object image from a first direction; a second image acquisition portion configured to acquire a second object image from a second direction different from the first direction; first illumination light emission portions provided on a straight line with the first image acquisition portion, the first image acquisition portion being interposed between the first illumination light emission portions, and arranged along a line orthogonal to an optical axis of the first image acquisition portion; and second illumination light emission portions lined up and arranged on a plane where the second image acquisition portion is arranged at the insertion portion, and a lining direction of the first illumination light emission portions and a lining direction of the second illumination light emission portions are arranged at positions to be a twisted relation.

### CROSS REFERENCE TO RELATED APPLICATION

This application is a continuation application of PCT/JP2015/079677 filed on Oct. 21, 2015 and claims benefit of Japanese Application No. 2014-240324 filed in Japan on Nov. 27, 2014, the entire contents of which are incorporated herein by this reference.

### BACKGROUND OF THE INVENTION

1. Field of the Invention

The present invention relates to an endoscope, and in particular, relates to an endoscope configured to acquire object images in a forward visual field and a sideward visual field.

2. Description of the Related Art

Conventionally, an endoscope has been widely used in a medical field and an industrial field. The endoscope includes illumination means and observation means on a distal end side of an insertion portion, and is inserted into a subject to perform observation and examination inside the subject.

In recent years, as disclosed in Japanese Patent Application Laid-Open Publication No. 2013-544617 for example, a multi-camera endoscope capable of observing two or more directions including a sideward visual field for which a side face side of an insertion portion is an observation visual field in addition to a forward visual field for which a forward side of the insertion portion is the observation visual field has been proposed. When such a multi-camera endoscope is used, an examiner can perform the observation inside a subject by a wide angle visual field by simultaneously observing two directions, forward and sideward.

### SUMMARY OF THE INVENTION

An endoscope of one aspect of the present invention includes: an insertion portion to be inserted into an inside of a subject in a longitudinal axis direction; a first image acquisition portion provided on a distal end face at a distal end of the insertion portion, and configured to acquire a first image from a first area including a forward direction of the insertion portion; a second image acquisition portion provided on the distal end face at the distal end of the insertion portion, and configured to acquire a second image from a second area including a sideward direction of the insertion portion; a first illumination light emission portion disposed on the distal end face at the distal end; and at least two second illumination light emission portions lined up and provided so that the second image acquisition portion is interposed between the second illumination light emission portions at an angle to the longitudinal axis on a peripheral side face at the distal end. The second image acquisition portion is arranged at a position where a plane passing through the first image acquisition portion and the first illumination light emission portion passes through, the second illumination light emission portions are provided away from the plane, and an optical axes of illumination light radiated respectively from the two second illumination light emission portions has a predetermined angle to a normal line at a point where the optical axis of illumination light passes through on an outer peripheral portion of the insertion portion and has an angle in a direction separating from an image pickup optical axis of an image made incident on the second image acquisition portion

An endoscope of another aspect of the present invention includes: an insertion portion to be inserted into an inside of a subject in a longitudinal axis direction; a first image acquisition portion provided on a distal end face of the insertion portion, and configured to acquire a first image from a first area including a forward direction of the insertion portion; a second image acquisition portion provided on a peripheral side face of the insertion portion, and configured to acquire a second image from a second area including a sideward direction of the insertion portion; a pair of first illumination light emission portions arranged with the first image acquisition portion being interposed between the pair of first illumination light emission portions along a line orthogonal to an optical axis of the first image acquisition portion, on the distal end face of the insertion portion; and two roughly L-shaped second illumination light emission portions disposed so as to surround the second image acquisition portion, on the peripheral side face of the insertion portion.

An endoscope system of one aspect in the present invention includes: an endoscope including an insertion portion to be inserted into an inside of a subject in a longitudinal axis direction, a first image acquisition portion provided on a distal end face at a distal end of the insertion portion, and configured to acquire a first image from a first area including a forward direction of the insertion portion, a second image acquisition portion provided on the distal end face at the distal end of the insertion portion, and configured to acquire a second image from a second area including a sideward direction of the insertion portion, a first illumination light emission portion disposed on the distal end face at the distal end, and at least two second illumination light emission portions lined up and provided so that the second image acquisition portion is interposed between the second illumination light emission portions at an angle to the longitudinal axis on a peripheral side face at the distal end, wherein the second image acquisition portion is arranged at a position where a plane passing through the first image acquisition portion and the first illumination light emission portion passes through, the second illumination light emission portions are provided away from the plane, and an optical axes of illumination light radiated respectively from the two second illumination light emission portions has a predetermined angle to a normal line at a point where the optical axis of illumination light passes through on an outer peripheral portion of the insertion portion and has an angle in a direction separating from an image pickup optical axis of an image made incident on the second image acquisition portion; an image processing portion configured to arrange the second image from the second image acquisition portion so as to be adjacent to the first image from the first image acquisition portion; and an image output portion configured to generate a display signal for causing a display portion to perform display, based on a signal relating to the first image and a signal relating to the second image from the image processing portion.

### BRIEF DESCRIPTION OF THE DRAWINGS

FIG. 1 is a configuration diagram illustrating a configuration of an endoscope system relating to a first embodiment;

FIG. 2 is a perspective view illustrating a configuration of a distal end section of an insertion portion of an endoscope relating to the first embodiment;

FIG. 3 is a side view illustrating the configuration of the distal end section of the insertion portion of the endoscope relating to the first embodiment;

FIG. 4 is a front view of a distal end portion of the endoscope for describing light distribution of illumination light relating to the first embodiment;

FIG. 5 is a diagram illustrating an illumination area by each illumination light in a sideward visual field relating to the first embodiment;

FIG. 6 is a perspective view illustrating the configuration of the distal end section of the insertion portion of the endoscope of a first modification relating to the first embodiment;

FIG. 7 is a side view illustrating the configuration of the distal end section of the insertion portion of the endoscope of the first modification relating to the first embodiment;

FIG. 8 is a diagram illustrating the illumination area by each illumination light in the sideward visual field of the first modification relating to the first embodiment;

FIG. 9 is a perspective view illustrating the configuration of the distal end section of the insertion portion of the endoscope of a second modification relating to the first embodiment;

FIG. 10 is a side view illustrating the configuration of the distal end section of the insertion portion of the endoscope of the second modification relating to the first embodiment;

FIG. 11 is a diagram illustrating the illumination area by each illumination light in the sideward visual field of the second modification relating to the first embodiment;

FIG. 12 is a front view of the distal end portion of the endoscope for describing an irradiation direction of the illumination light of a third modification relating to the first embodiment;

FIG. 13 is a perspective view illustrating the configuration of the distal end section of the insertion portion of the endoscope of a fourth modification relating to the first embodiment;

FIG. 14 is a side view illustrating the configuration of the distal end section of the insertion portion of the endoscope of the fourth modification relating to the first embodiment;

FIG. 15 is a perspective view illustrating the configuration of the distal end section of the insertion portion of the endoscope relating to a second embodiment;

FIG. 16 is a sectional view of a light guide portion and a light guide along an XVI-XVI line in FIG. 15 relating to the second embodiment;

FIG. 17 is a sectional view of the light guide portion and the light guide relating to the second embodiment;

FIG. 18 is a plan view for describing a display example of an endoscopic image by three monitors relating to a third embodiment;

FIG. 19 is a plan view for describing one example of a display state of the three monitors relating to the third embodiment;

FIG. 20 is a plan view illustrating one example of an installation state of the three monitors relating to the third embodiment; and

FIG. 21 is a perspective view of the distal end portion of the insertion portion to which a side observation unit is attached relating to a fourth embodiment.

### DETAILED DESCRIPTION OF THE PREFERRED EMBODIMENTS

Hereinafter, an endoscope apparatus which is the present invention will be described. Note that, in following description, drawings based on individual embodiments are schematic, it should be noted that a relation between a thickness and a width of individual sections and a ratio of the thicknesses of the respective sections or the like are different from the actual ones, and even between the drawings, a section where the relation of mutual dimensions or the ratio is different is sometimes included.

Note that, for an endoscope in the following configuration description, a so-called flexible endoscope, an insertion portion of which is flexible to be inserted into a digestive organ at an upper portion or a lower portion of a living body, will be described as an example; however, without being limited to the flexible scope, a technology is applicable also to a so-called rigid endoscope, the insertion portion of which is rigid, used for a surgery.

### First Embodiment

First, an endoscope system of one aspect of the present invention will be described based on the drawings.

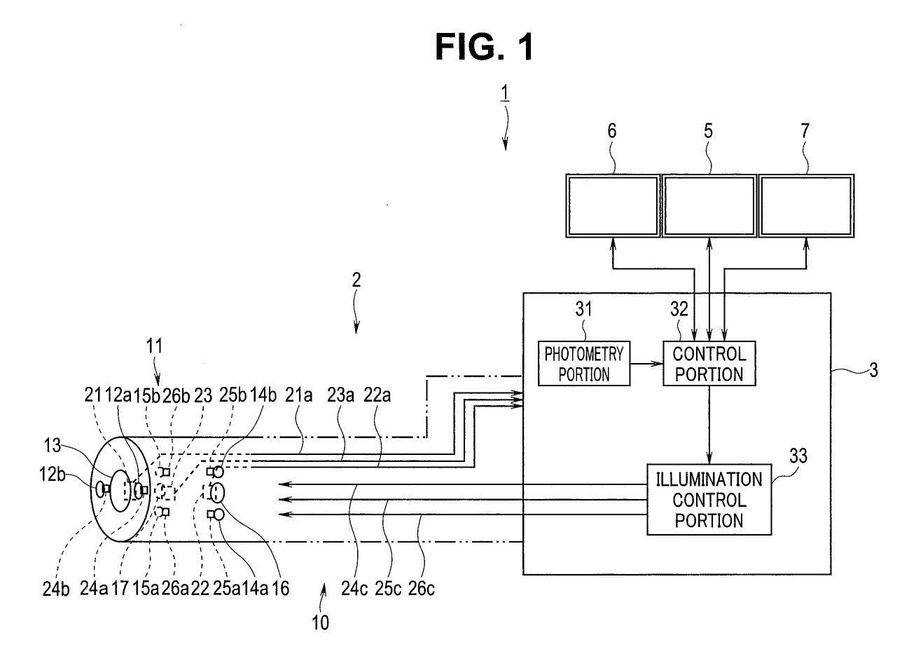

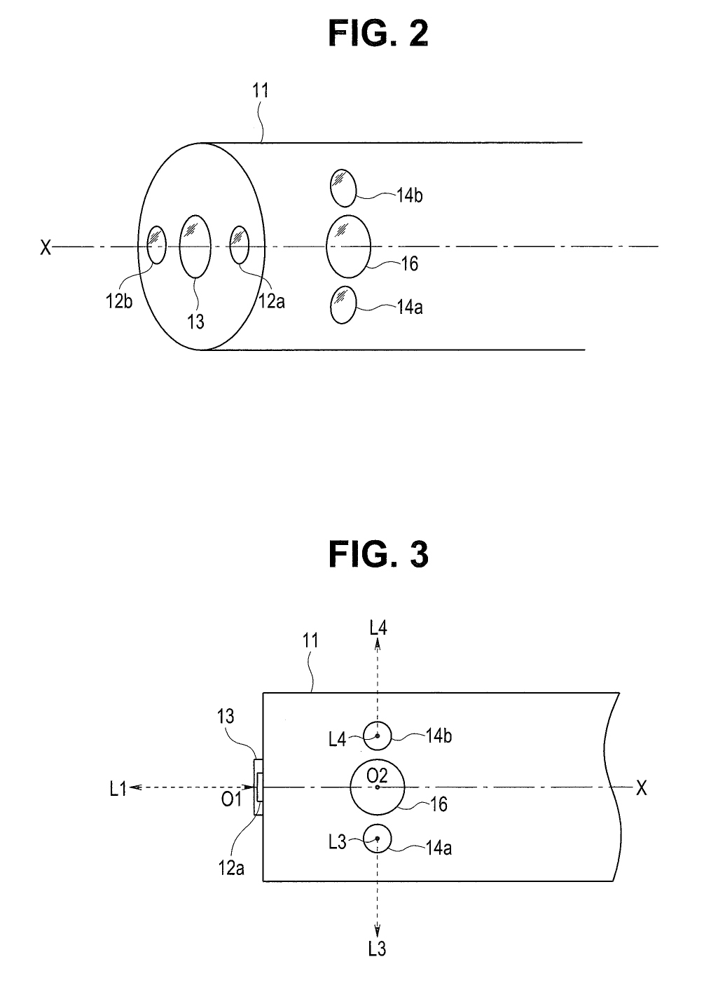

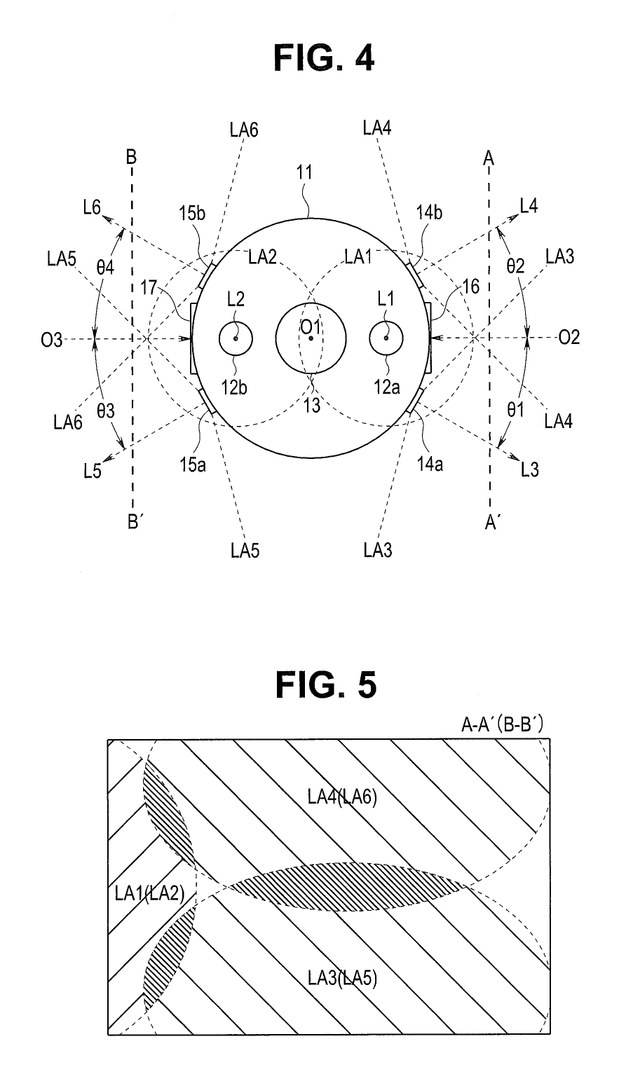

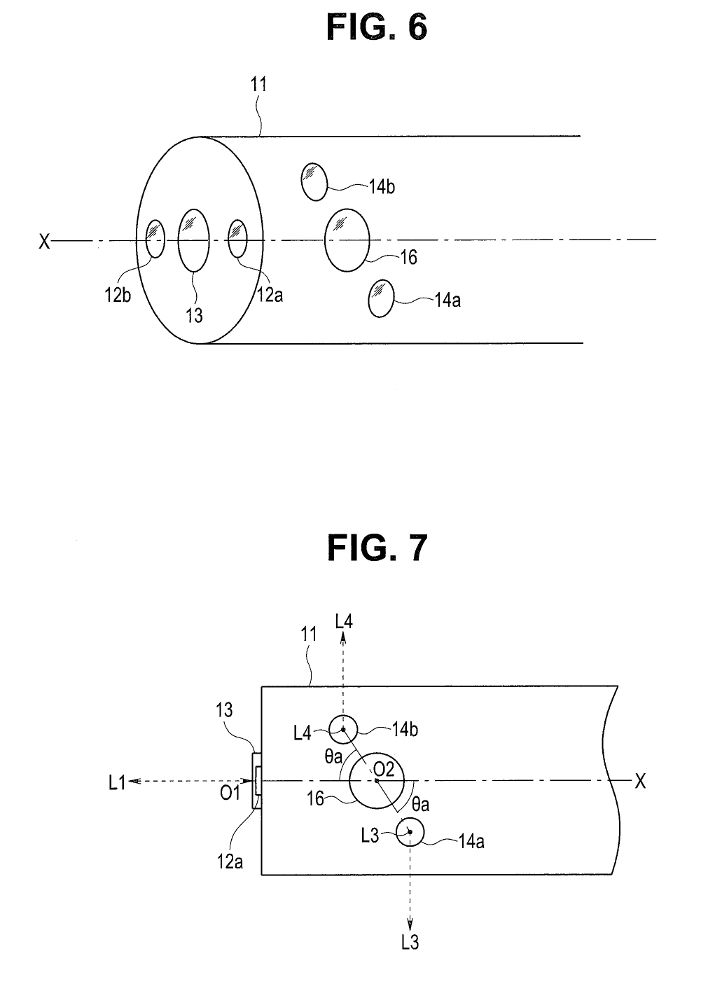

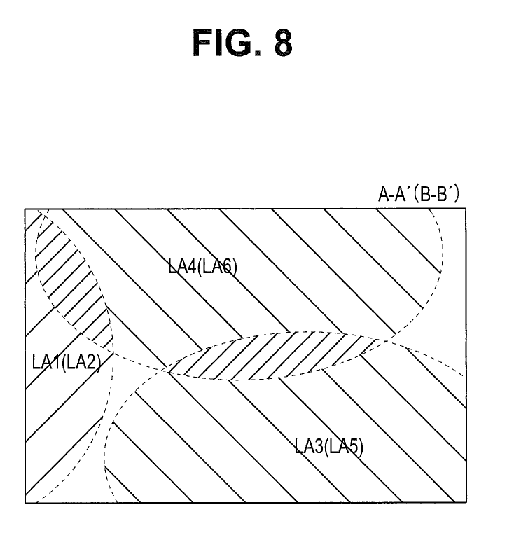

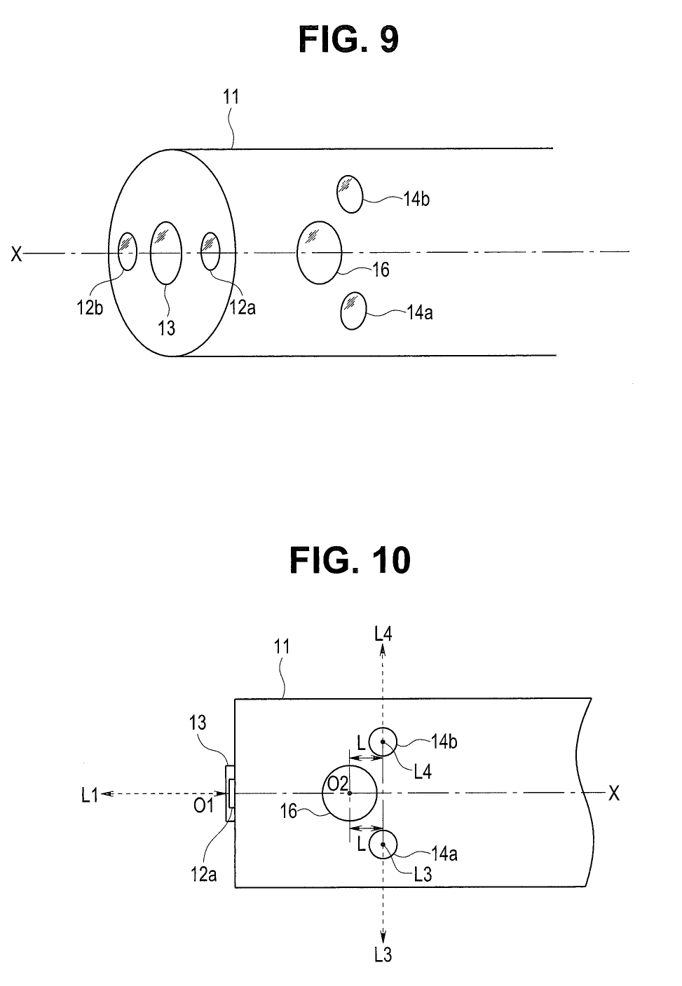

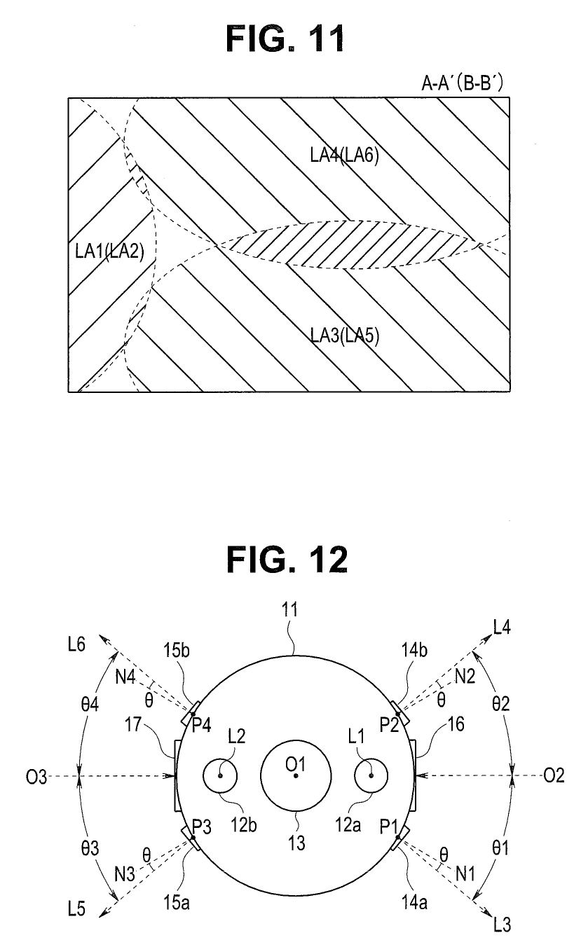

FIG. 1 is a configuration diagram illustrating a configuration of the endoscope system relating to the present embodiment, FIG. 2 is a perspective view illustrating a configuration of a distal end section of an insertion portion of an endoscope, FIG. 3 is a side view illustrating the configuration of the distal end section of the insertion portion of the endoscope, FIG. 4 is a front view of a distal end portion of the endoscope for describing light distribution of illumination light, FIG. 5 is a diagram illustrating an illumination area by each illumination light in a sideward visual field, FIG. 6 is a perspective view illustrating the configuration of the distal end section of the insertion portion of the endoscope of a first modification, FIG. 7 is a side view illustrating the configuration of the distal end section of the insertion portion of the endoscope of the first modification, FIG. 8 is a diagram illustrating the illumination area by each illumination light in the sideward visual field of the first modification, FIG. 9 is a perspective view illustrating the configuration of the distal end section of the insertion portion of the endoscope of a second modification, FIG. 10 is a side view illustrating the configuration of the distal end section of the insertion portion of the endoscope of the second modification, FIG. 11 is a diagram illustrating the illumination area by each illumination light in the sideward visual field of the second modification, FIG. 12 is a front view of the distal end portion of the endoscope for describing an irradiation direction of the illumination light of a third modification,

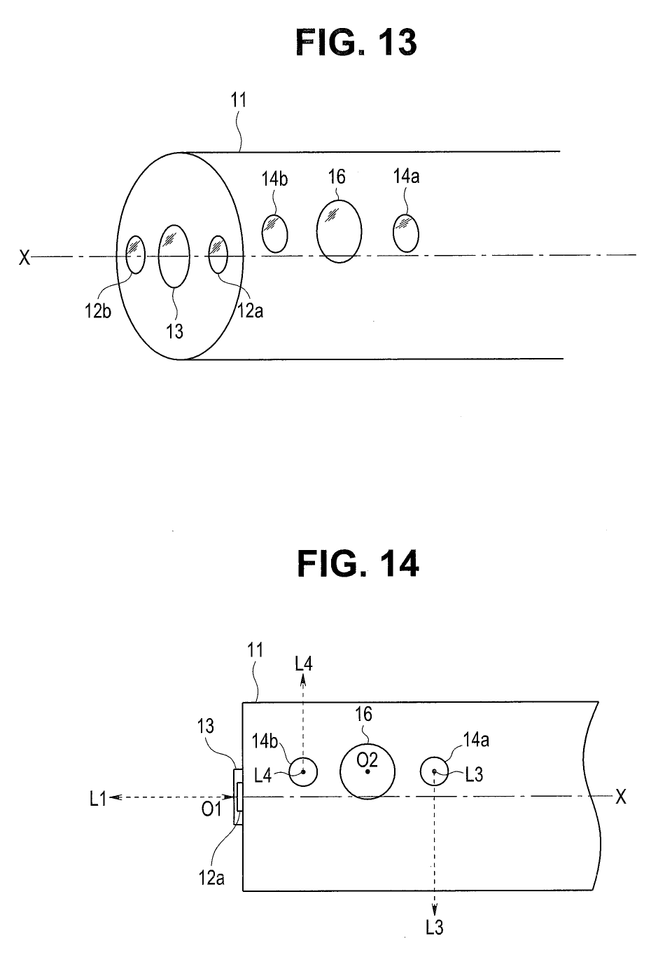

FIG. 13 is a perspective view illustrating the configuration of the distal end section of the insertion portion of the endoscope of a fourth modification, and FIG. 14 is a side view illustrating the configuration of the distal end section of the insertion portion of the endoscope of the fourth modification.

An endoscope system 1 illustrated in FIG. 1 is configured including an endoscope 2, a processor 3, and display devices 5, 6 and 7 as three monitors here.

The endoscope 2 includes a flexible insertion portion 10 to be inserted into an inside of a subject, and an operation portion not shown in the figure, and is connected to the processor 3 by an endoscope cable not shown in the figure.

The endoscope 2 is provided with two illumination windows 12a and 12b to be an illumination light emission portion and an observation window 13 for a forward visual field, and four illumination windows 14a, 14b, 15a and 15b to be the illumination light emission portion and two observation windows 16 and 17 for a sideward visual field on a distal end portion 11 of the insertion portion 10.

That is, the endoscope 2 includes the four illumination windows 14a, 14b, 15a and 15b here in addition to the two illumination windows 12a and 12b, and includes the two observation windows 16 and 17 here in addition to the observation window 13.

The two illumination windows 12a and 12b and the observation window 13 are for the forward visual field.

Furthermore, the two illumination windows 14a and 14b and the observation window 16 are for a first sideward visual field, and the two illumination windows 15a and 15b and the observation window 17 are for a second sideward visual field.

The two illumination windows 14a and 14b for the first sideward visual field are provided on one side portion of the distal end portion 11 with the observation window 16 being interposed between the two illumination windows 14a and 14b, on a left side portion viewing the insertion portion 10 toward a direction of the distal end portion 11 here.

In addition, the two illumination windows 15a and 15b for the second sideward visual field are provided on the other side portion of the distal end portion 11 with the observation window 17 being interposed between the two illumination windows 15a and 15b, on a right side portion viewing the insertion portion 10 toward the direction of the distal end portion 11 here.

Then, the plurality of, two here, observation windows 16 and 17 are arranged at a roughly uniform angle in a circumferential direction of the insertion portion 10.

That is, the distal end portion 11 of the insertion portion 10 includes a distal end rigid member not shown in the figure, the illumination windows 12a and 12b and the observation window 13 are provided on a distal end face of the distal end rigid member, and the two illumination windows 14a and 14b forming a pair and the observation window 16 and the two illumination windows 15a and 15b forming a pair and the observation window 17 are provided on a side face portion of the distal end rigid member.

Inside the distal end portion 11, on a rear side of the observation window 13 for the forward visual field, an image pickup unit 21 for the forward visual field is disposed. In addition, inside the distal end portion 11, an image pickup unit 22 for the first sideward visual field is disposed on the rear side of the observation window 16, and an image pickup unit 23 for the second sideward visual field is disposed on the rear side of the observation window 17.

Each of the three image pickup units 21, 22 and 23 that are image pickup portions includes an image pickup device such as a CCD or a CMOS, is electrically connected with the processor 3, is controlled by the processor 3, and outputs an image pickup signal to the processor 3. Therefore, the respective image pickup units 21, 22 and 23 configure the image pickup portions that photoelectrically convert an object image.

Then, the observation window 13 is arranged in a direction of inserting the insertion portion 10 at the distal end portion 11 of the insertion portion 10, and the observation windows 16 and 17 are arranged in an outer diameter direction of the insertion portion 10 at the side face portion of the insertion portion 10.

That is, the observation window 13 configures a first image acquisition portion provided on the distal end portion 11 of the insertion portion 10 and configured to acquire a first image which is the object image from a first area including a forward direction of the insertion portion roughly parallel to a longitudinal direction of the insertion portion 10.

In addition, each of the observation windows 16 and 17 configures a second image acquisition portion provided on the distal end portion 11 of the insertion portion 10 and configured to acquire a second image which is the object image from an area that crosses the longitudinal direction of the insertion portion 10 at a certain first angle and is different from the forward direction, a second area including a lateral direction orthogonal to the longitudinal direction of the insertion portion 10 here for example.

In other words, the first image is the object image of the first area including the forward direction of the insertion portion roughly parallel to the longitudinal direction of the insertion portion 10, and the second image is the object image of the second area including the sideward direction of the insertion portion crossing the longitudinal direction of the insertion portion 10.

Note that a second direction may be a vertical direction provided on positions for which the positions of the observation windows 16 and 17 are rotated about 90 degrees around a center axis of the insertion portion 10.

On the rear side of the illumination windows 12a and 12b for the forward visual field, illumination light emitting elements 24a and 24b for the forward visual field are disposed respectively inside the distal end portion 11.

In addition, on the rear side of the illumination windows 14a and 14b, illumination light emitting elements 25a and 25b for the first sideward visual field are disposed respectively inside the distal end portion 11.

Then, on the rear side of the illumination windows 15a and 15b, illumination light emitting elements 26a and 26b for the second sideward visual field are disposed respectively inside the distal end portion 11.

The illumination light emitting elements 24a, 24b, 25a, 25b, 26a and 26b are light emitting diodes (LEDs) for example.

Therefore, the two illumination windows 12a and 12b corresponding to the illumination light emitting element 24a are illumination portions configured to emit illumination light in the forward direction, and the illumination windows 14a, 14b, 15a and 15b corresponding to each of the illumination light emitting elements 25a, 25b, 26a and 26b are illumination portions configured to emit the illumination light in the sideward direction.

In the processor 3, a photometry portion 31, a control portion 32, and an illumination control portion 33 are incorporated. The control portion 32 receives input of three image pickup signals outputted from the three image pickup units 21, 22 and 23 through image pickup cables 21a, 22a and 23a, generates and combines three endoscopic images based on the three image pickup signals, and outputs the images to the three display devices 5, 6 and 7.

Note that the illumination control portion 33 provided in the processor 3 is controlled by the control portion 32.

The illumination control portion 33 is a circuit configured to control a light emission quantity and ON/OFF of the illumination light emitting elements 24a, 24b, 25a, 25b, 26a and 26b, and outputs a control signal through signal lines 24c, 25c and 26c for the respective illumination light emitting elements 24a, 24b, 25a, 25b, 26a and 26b.

Then, the illumination control portion 33 controls a light quantity of the respective illumination light emitting elements 24a, 24b, 25a, 25b, 26a and 26b based on a light adjustment signal from the control portion 32.

Further, the illumination control portion 33 controls a light emission timing of ON and OFF of the respective illumination light emitting elements. That is, the illumination control portion 33 configures the illumination control portion configured to control emission of the illumination light in the forward direction and the emission of the illumination in the sideward direction at mutually different predetermined timing.

The endoscope system 1 configured as described above displays the object image acquired by the respective image pickup units 21, 22 and 23 provided on the distal end portion 11 of the endoscope 2 at the three display devices 5, 6 and 7.

Next, an arrangement relation of the two illumination windows 12a and 12b for the forward visual field and the four illumination windows 14a, 14b, 15a and 15b for the sideward visual field disposed at the distal end portion 11 will be described below.

As illustrated in FIG. 2 to FIG. 4, the two illumination windows 12a and 12b for the forward visual field are disposed on the distal end face of the distal end portion 11.

The two illumination windows 12a and 12b are, as described above, lined up and arranged in the lateral direction orthogonal to a longitudinal axis X of the insertion portion 10 in a front view of the distal end portion 11, with the observation window 13 being interposed between the illumination windows 12a and 12b.

Note that the two illumination windows 12a and 12b here are provided on one straight line with the observation window 13 with the observation window 13 being interposed between the illumination windows 12a and 12b, on the positions point-symmetrical to a center of the observation window 13 for example.

In contrast, the four illumination windows 14a, 14b, 15a and 15b for the sideward visual field are disposed at an outer peripheral side face portion of the distal end portion 11.

Of the four illumination windows 14a, 14b, 15a and 15b, the two illumination windows 14a and 14b are lined up and provided in the vertical direction perpendicular to the longitudinal axis X of the insertion portion 10 at one peripheral side face portion of the distal end portion 11.

Note that the two illumination windows 14a and 14b here are disposed at positions point-symmetrical to the center of the observation window 16 for example with the observation window 16 being interposed between the illumination windows 14a and 14b in the vertical direction.

In addition, the two illumination windows 15a and 15b are lined up in the vertical direction perpendicular to the longitudinal axis X of the insertion portion 10 at the other peripheral side face portion of the distal end portion 11, specifically lined up and provided with the observation window 17 being interposed between the illumination windows 15a and 15b in the vertical direction (not shown in FIG. 2 and FIG. 3). Note that the two illumination windows 15a and 15b here are disposed at the positions point-symmetrical to the center of the observation window 17.

From the above, in the present embodiment, a lining direction of the two illumination windows 12a and 12b which are first illumination light emission portions and a lining direction of the two illumination windows 14a and 14b or a lining direction of the two illumination windows 15a and 15b which are second illumination light emission portions are arranged at the positions to be a twisted relation, geometrically not existing on the same plane.

Incidentally, as illustrated in FIG. 3 and FIG. 4, an image pickup optical axis O1 of the object image from the forward direction is made incident on the observation window 13 for the forward visual field, and image pickup optical axes O2 and O3 of the object image from one or the other side are made incident on the observation windows 16 and 17 for the sideward visual field (in FIG. 3, the two illumination windows 15a and 15b and the observation window 17 are not shown).

Then, the two illumination windows 12a and 12b for the forward visual field radiate the illumination light of illumination optical axes L1 and L2 toward an object in the forward direction in irradiation ranges LA1 and LA2 of predetermined light intensity.

In contrast, the four illumination windows 14a, 14b, 15a and 15b for the sideward visual field radiate the illumination light of illumination optical axes L3, L4, L5 and L6 toward the object at the side in irradiation ranges LA3, LA4, LA5 and LA6 of the predetermined light intensity.

Here, the two illumination windows 14a and 14b for one sideward visual field radiate the illumination light of the illumination optical axes L3 and L4 in directions separating from the image pickup optical axis O2 of the object image made incident on the observation window 16 at predetermined angles θ1 and θ2. Note that the predetermined angles θ1 and θ2 here are roughly identical angles (θ1≈θ2).

Furthermore, the two illumination windows 15a and 15b for the other sideward visual field also radiate the illumination light of the illumination optical axes L5 and L6 in the directions separating from the image pickup optical axis O3 of the object image made incident on the observation window 17 at predetermined angles θ3 and θ4. Note that the predetermined angles θ3 and θ4 here are also the roughly identical angles (θ3≈θ4).

Further, here, the predetermined angles θ1 and θ2 and the predetermined angles θ3 and θ4 are also the roughly identical angles (θ1≈θ2≈θ3≈θ4). Note that the predetermined angles θ1, θ2, θ3 and θ4 can be appropriately set.

The endoscope 2 configured as described above can suppress generation of illumination irregularities by reducing overlapping parts of the irradiation ranges LA1 (LA2), LA3 (LA4) and LA5 (LA6) to be light distribution ranges of predetermined light intensity as illustrated in FIG. 5, in illumination light distribution of an image pickup area on a plane at the position of a dotted line A-A′ at one side close to the observation window 16 or an image pickup area on a plane at the position of a dotted line B-B′ at the other side close to the observation window 17 for example illustrated in FIG. 4.

In detail, in the image pickup area in one sideward visual field direction close to the observation window 16, the illumination light in the irradiation range LA1 of the predetermined light intensity radiated from the illumination window 12a to the forward visual field irradiates a forward side (a left side of page space) of the image pickup area made incident on the observation window 16.

In addition, the illumination light in the irradiation range LA3 of the predetermined light intensity radiated from the illumination window 14a to a lower part of one sideward visual field irradiates a lower side of the image pickup area made incident on the observation window 16.

Further, the illumination light in the irradiation range LA4 of the predetermined light intensity radiated from the illumination window 14b to an upper part of one sideward visual field irradiates an upper side of the image pickup area made incident on the observation window 16.

Then, each illumination light emitted from the respective illumination windows 12a, 14a and 14b is radiated to the almost entire image pickup area close to the observation window 16 with a small overlapping range of the irradiation ranges LA1, LA3 and LA4 of the predetermined light intensity.

Similarly, in the image pickup area in the other sideward visual field direction close to the observation window 17, the illumination light in the irradiation range LA2 of the predetermined light intensity radiated from the illumination window 12b to the forward visual field irradiates the forward side (the left side of page space) of the image pickup area made incident on the observation window 17.

In addition, the illumination light in the irradiation range LA5 of the predetermined light intensity radiated from the illumination window 15a to the lower part of the other sideward visual field irradiates the lower side of the image pickup area made incident on the observation window 17.

Further, the illumination light in the irradiation range LA6 of the predetermined light intensity radiated from the illumination window 15b to the upper part of the other sideward visual field irradiates the upper side of the image pickup area made incident on the observation window 17.

Then, each illumination light emitted from the respective illumination windows 12b, 15a and 15b is radiated to the almost entire image pickup area close to the observation window 17 with the small overlapping range of the irradiation ranges LA2, LA5 and LA6 of the predetermined light intensity.

In this way, for the endoscope 2 of the present embodiment, the illumination windows 14a and 14b or the illumination windows 15a and 15b forming the pair for the sideward visual field are lined up and arranged with the observation window 16 or the observation window 17 being interposed between the illumination windows in the vertical direction orthogonal to the longitudinal axis X of the insertion portion 10 at the peripheral side face portion of the distal end portion 11.

Thus, the lining direction of the illumination windows 14a and 14b or the lining direction of the illumination windows 15a and 15b is arranged to be the twisted relation with the lining direction of the two illumination windows 12a and 12b for the forward visual field, bias of the illumination light is prevented and the generation of the illumination irregularities can be suppressed.

Further, by the configuration of radiating the illumination light of the illumination optical axes L3 and L4 or the illumination optical axes L5 and L6 in the directions separating from the image pickup optical axis O2 or the image pickup optical axis O3 of the object image made incident on the observation window 16 or the observation window 17 at the predetermined angles θ1 and θ2 or the predetermined angles θ3 and θ4, the generation of the illumination irregularities in the illumination light can be suppressed further, even when a side portion of the distal end portion 11 becomes close to a luminal wall without increasing a number of the illumination windows for the sideward visual field.

According to the description above, since the generation of the illumination irregularities can be suppressed by the arrangement of illumination means by the six illumination windows 12a, 12b, 14a, 14b, 15a and 15b of the same number as the conventional illumination windows and the six illumination light emitting elements 24a, 24b, 25a, 25b, 26a and 26b, the endoscope 2 can prevent enlargement of the distal end portion 11 of the insertion portion 10 and complication of an internal structure together.

### First Modification

Note that the endoscope 2 may be configured as illustrated in FIG. 6 and FIG. 7.

Specifically, of the four illumination windows 14a, 14b, 15a and 15b for the sideward visual field here, the two illumination windows 14a and 14b are juxtaposed with the observation window 16 being interposed between the illumination windows 14a and 14b in the vertical direction at a predetermined angle θa (45° for example) to the longitudinal axis X of the insertion portion 10, at one peripheral side face portion of the distal end portion 11.

Then, the two illumination windows 14a and 14b are also disposed at the positions point-symmetrical to the center of the observation window 16 here.

Note that, here, the configuration is that the illumination window 14a is positioned on a proximal end side (rear side) of the insertion portion 10 and the illumination window 14b is positioned on a distal end side (forward side) of the insertion portion 10; however, without being limited to the configuration, the illumination window 14a may be arranged at the front and the illumination window 14b may be arranged at the rear.

Further, though not shown in the figure here, the two illumination windows 15a and 15b are also juxtaposed with the observation window 17 being interposed between the illumination windows 15a and 15b in the vertical direction at the predetermined angle θa (45° for example) to the longitudinal axis X of the insertion portion 10, at the other peripheral side face portion of the distal end portion 11 (not shown in FIG. 6 and FIG. 7).

Note that the two illumination windows 15a and 15b are also disposed at the positions point-symmetrical to the center of the observation window 17 here.

From the above, also in the present embodiment, the lining direction of the two illumination windows 12a and 12b which are the first illumination light emission portions and the lining direction of the two illumination windows 14a and 14b or the lining direction of the two illumination windows 15a and 15b which are the second illumination light emission portions are arranged at the positions to be the twisted relation, geometrically not existing on the same plane.

Also in the endoscope 2 of the present modification, the generation of the illumination irregularities can be suppressed by reducing the overlapping parts of the illumination light in the irradiation ranges LA1 (LA2), LA3 (LA4) and LA5 (LA6) to be the light distribution ranges of the predetermined light intensity radiated from the respective illumination windows 12a, 12b, 14a, 14b, 15a and 15b as illustrated in FIG. 8, in the illumination light distribution of the image pickup area on the plane at the position of the dotted line A-A′ at one side close to the observation window 16 or the image pickup area on the plane at the position of the dotted line B-B′ at the other side close to the observation window 17 for example illustrated in FIG. 4.

Also in this way, each illumination light emitted from the respective illumination windows 12a, 14a and 14b is radiated to the almost entire image pickup area close to the observation window 16 with the small overlapping range of the irradiation ranges LA1, LA3 and LA4 of the predetermined light intensity.

Similarly, each illumination light emitted from the respective illumination windows 12b, 15a and 15b is also radiated to the almost entire image pickup area close to the observation window 17 with the small overlapping range of the irradiation ranges LA2, LA5 and LA6 of the predetermined light intensity.

Also by the endoscope 2 of the present modification configured as described above, in addition to effects described above, the illumination light can be radiated to a subject in a wide range further.

### Second Modification

In addition, the endoscope 2 may be configured as illustrated in FIG. 9 and FIG. 10.

Specifically, of the four illumination windows 14a, 14b, 15a and 15b for the sideward visual field here, the two illumination windows 14a and 14b are juxtaposed with the observation window 16 being interposed between the illumination windows 14a and 14b in the vertical direction orthogonal to the longitudinal axis X of the insertion portion 10, at one peripheral side face portion of the distal end portion 11.

Then, the two illumination windows 14a and 14b are disposed such that the respective centers are positioned on the proximal end side (rear side) of the insertion portion 10 by a predetermined distance L from the center of the observation window 16.

Further, though not shown in the figure here, the two illumination windows 15a and 15b are also juxtaposed with the observation window 17 being interposed between the illumination windows 15a and 15b in the vertical direction orthogonal to the longitudinal axis X of the insertion portion 10, at the other peripheral side face portion of the distal end portion 11 (not shown in FIG. 9 and FIG. 10).

Then, the two illumination windows 15a and 15b are disposed such that the respective centers are positioned on the proximal end side (rear side) of the insertion portion 10 by the predetermined distance L from the center of the observation window 17.

From the above, also in the present embodiment, the lining direction of the two illumination windows 12a and 12b which are the first illumination light emission portions and the lining direction of the two illumination windows 14a and 14b or the lining direction of the two illumination windows 15a and 15b which are the second illumination light emission portions are arranged at the positions to be the twisted relation, geometrically not existing on the same plane.

Also in the endoscope 2 of the present modification, the generation of the illumination irregularities can be suppressed by reducing the overlapping parts of the illumination light in the irradiation ranges LA1 (LA2), LA3 (LA4) and LA5 (LA6) to be the light distribution ranges of the predetermined light intensity radiated from the respective illumination windows 12a, 12b, 14a, 14b, 15a and 15b as illustrated in FIG. 11, in the illumination light distribution of the image pickup area on the plane at the position of the dotted line A-A′ at one side close to the observation window 16 or the image pickup area on the plane at the position of the dotted line B-B′ at the other side close to the observation window 17 for example illustrated in FIG. 4.

Also in this way, each illumination light emitted from the respective illumination windows 12a, 14a and 14b is radiated to the almost entire image pickup area close to the observation window 16 with the small overlapping range of the irradiation ranges LA1, LA3 and LA4 of the predetermined light intensity.

Similarly, each illumination light emitted from the respective illumination windows 12b, 15a and 15b is also radiated to the almost entire image pickup area close to the observation window 17 with the small overlapping range of the irradiation ranges LA2, LA5 and LA6 of the predetermined light intensity.

Also by the endoscope 2 of the present modification configured as described above, in addition to the effects described above, the illumination light can be radiated to the subject in the wide range further.

### Third Modification

Note that, after the lining direction of the two illumination windows 12a and 12b and the lining direction of the two illumination windows 14a and 14b or the lining direction of the two illumination windows 15a and 15b are arranged to be the twisted relation as in the first embodiment, the first modification and the second modification, it is conceivable to arrange the illumination optical axes of the two illumination windows 14a and 14b or the two illumination windows 15a and 15b as follows, as illustrated in FIG. 12.

It is preferable to set the two illumination windows 14a and 14b for one sideward visual field such that the illumination optical axes L3 and L4 of the illumination light that are set in the directions of separating from the image pickup optical axis O2 of the object image made incident on the observation window 16 having the predetermined angles θ1 and θ2 are set in the directions further separating from the image pickup optical axis O2 at a predetermined angle θ to normal lines N1 and N2 at points P1 and P2 on the outer peripheral portion of the distal end portion 11 where the illumination optical axes L3 and L4 respectively pass through.

Similarly, it is preferable to set the two illumination windows 15a and 15b for the other sideward visual field also in the directions further separating from the image pickup optical axis O3 at the predetermined angle θ to normal lines N3 and N4 at points P3 and P4 on the outer peripheral portion of the distal end portion 11 where the illumination optical axes L5 and L6 of the illumination light set in the directions of separating from the image pickup optical axis O3 of the object image made incident on the observation window 17 at the predetermined angles θ3 and θ4 respectively pass through.

By such a configuration, the overlapping parts of each illumination light radiated in the sideward visual field direction can be reduced more, the illumination irregularities can be reduced, and brightness can be made uniform.

### Fourth Modification

Note that, as long as the lining direction of the two illumination windows 12a and 12b which are the first illumination light emission portions and the lining direction of the two illumination windows 14a and 14b or the lining direction of the two illumination windows 15a and 15b which are the second illumination light emission portions are arranged at the positions to be the twisted relation, geometrically not existing on the same plane as in the respective embodiments described above, a following form may be sufficient.

Specifically, of the four illumination windows 14a, 14b, 15a and 15b for the sideward visual field here, the two illumination windows 14a and 14b are juxtaposed at the positions which are in the direction parallel to the longitudinal axis X of the insertion portion 10 and shifted in the direction vertical to the longitudinal axis X, as in FIG. 13 and FIG. 14, at one peripheral side face portion of the distal end portion 11.

Note that, here, the configuration is that the illumination window 14a is positioned on the proximal end side (rear side) of the insertion portion 10 and the illumination window 14b is positioned on the distal end side (forward side) of the insertion portion 10; however, without being limited to the configuration, the illumination window 14a may be arranged at the front and the illumination window 14b may be arranged at the rear.

Further, though not shown in the figure here, the two illumination windows 15a and 15b are also juxtaposed at the positions which are in the direction parallel to the longitudinal axis X of the insertion portion 10 and shifted in the direction vertical to the longitudinal axis X, at the other peripheral side face portion of the distal end portion 11.

Also in such an endoscope 2, the generation of the illumination irregularities can be suppressed by reducing the overlapping parts of the illumination light in the irradiation ranges LA1 (LA2), LA3 (LA4) and LA5 (LA6) to be the light distribution ranges of the predetermined light intensity radiated from the respective illumination windows 12a, 12b, 14a, 14b, 15a and 15b, in the illumination light distribution of the image pickup area on the plane at the position of the dotted line A-A′ at one side close to the observation window 16 or the image pickup area on the plane at the position of the dotted line B-B′ at the other side close to the observation window 17 for example illustrated in FIG. 4.

Also in this way, each illumination light emitted from the respective illumination windows 12a, 14a and 14b is radiated to the almost entire image pickup area close to the observation window 16 with the small overlapping range of the irradiation ranges LA1, LA3 and LA4 of the predetermined light intensity.

Similarly, each illumination light emitted from the respective illumination windows 12b, 15a and 15b is also radiated to the almost entire image pickup area close to the observation window 17 with the small overlapping range of the irradiation ranges LA2, LA5 and LA6 of the predetermined light intensity.

Also by the endoscope 2 of the present modification configured as described above, in addition to the effects described above, the illumination light can be radiated to the subject in the wide range further.

### Second Embodiment

Next, the endoscope system of the second embodiment of the present invention will be described hereinafter based on the drawings. Note that, in the following description, same signs are used for identical components described in the first embodiment described above, and detailed description of the components is omitted.

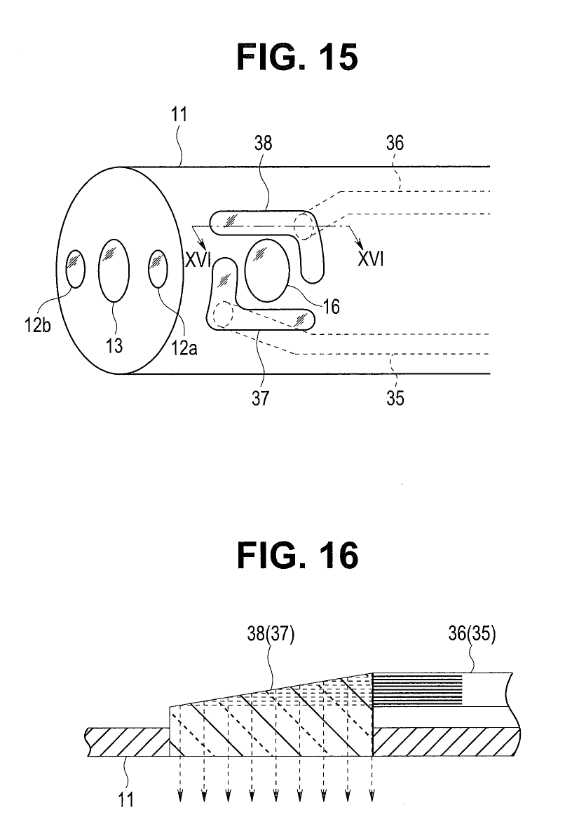

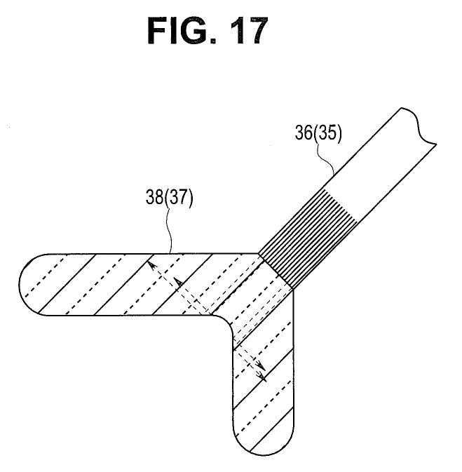

FIG. 15 is a perspective view illustrating the configuration of the distal end section of the insertion portion of the endoscope, FIG. 16 is a sectional view of a light guide portion and a light guide along an XVI-XVI line in FIG. 15, and FIG. 17 is a sectional view of the light guide portion and the light guide.

For the endoscope 2 of the present embodiment, as illustrated in FIG. 15, roughly L-shaped light guide portions 37 and 38 as the two illumination windows are disposed so as to surround the observation window 16 at one side portion of the distal end portion 11 of the insertion portion 10. Note that, though not shown in the figure, similarly, the light guide portions 37 and 38 are provided so as to surround the observation window 17 at one side portion of the distal end portion 11 of the insertion portion 10.

Further, the endoscope 2 here is configured to transmit the illumination light to the respective light guide portions 37 and 38 by light guide bundles 35 and 36.

Then, for the endoscope 2, as illustrated in FIG. 16 and FIG. 17, end faces of the light guide bundles 35 and 36 are connected to butt against the light guide portions 37 and 38, the light guide portions 37 and 38 reflect and diffuse the illumination light made incident from the light guide bundles 35 and 36, and thus more uniform illumination light can be radiated in the sideward visual field direction.

That is, the light guide portions 37 and 38 configure so-called surface light sources using a light diffusing film for a light guiding plate and a diffusion plate or the like.

Note that, the configuration is such that the light guide bundles 35 and 36 are used here; however, without being limited to the configuration, the illumination light emitting element such as a light emitting diode (LED) may be used similarly to the first embodiment.

It is preferable that the lining direction of parts that the light guide bundles 35 and 36 respectively guide the light to the light guide portions 37 and 38 is arranged at the position to be the twisted relation, geometrically not existing on the same plane, with the lining direction of the two illumination windows 12a and 12b.

In this way, for the endoscope 2 of the present embodiment, by providing the light guide portions 37 and 38 which are the surface light sources so as to surround the observation windows 16 and 17 for the sideward visual field, an irradiation surface of the illumination light becomes wide, and peripheries of the observation windows 16 and 17 can be illuminated by uniform predetermined light intensity.

As a result, the endoscope 2 of the present embodiment can be also configured to suppress the generation of the illumination irregularities more, similarly to the first embodiment.

### Modification

Note that, for the light guide portions 37 and 38, by a fluorescent material such as zinc sulfide or a fluorescent paint, a diffusion effect may be improved and a structure may be simplified. Further, even when supply of the illumination light from the light guide bundles 35 and 36 is stopped, the light guide portions 37 and 38 continue to emit the light for a constant period of time, leading to reduction of power consumption.

### Third Embodiment

Next, the endoscope system of the third embodiment of the present invention will be described hereinafter based on the drawings. Note that, in the following description, the same signs are used for the identical components described in the first embodiment described above, and the detailed description of the components is omitted.

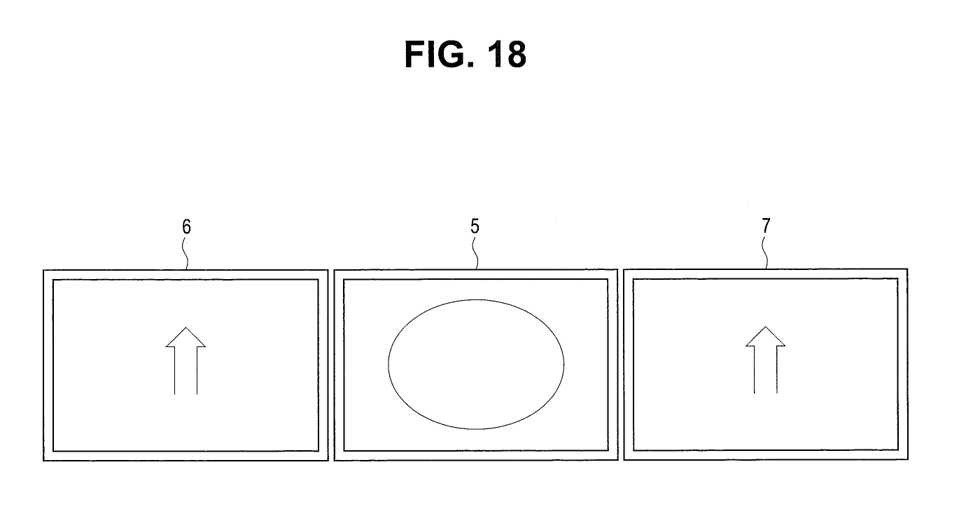

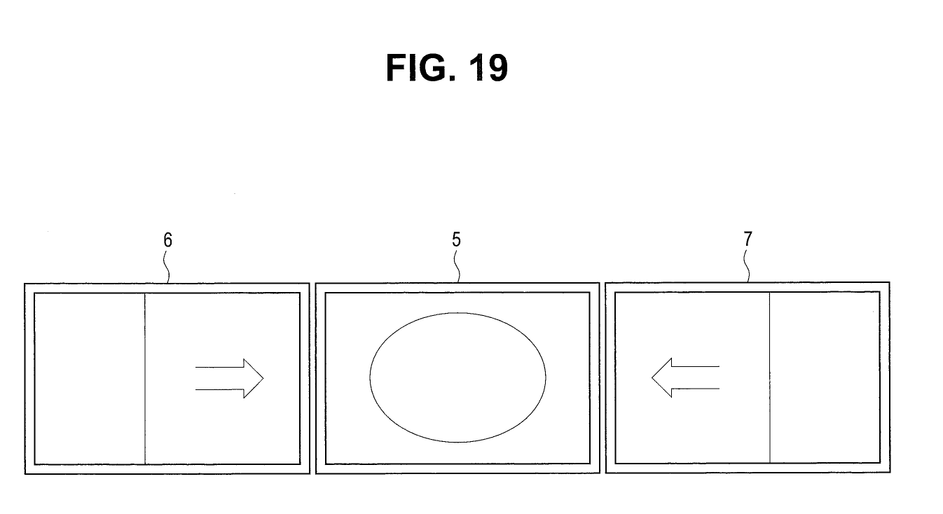

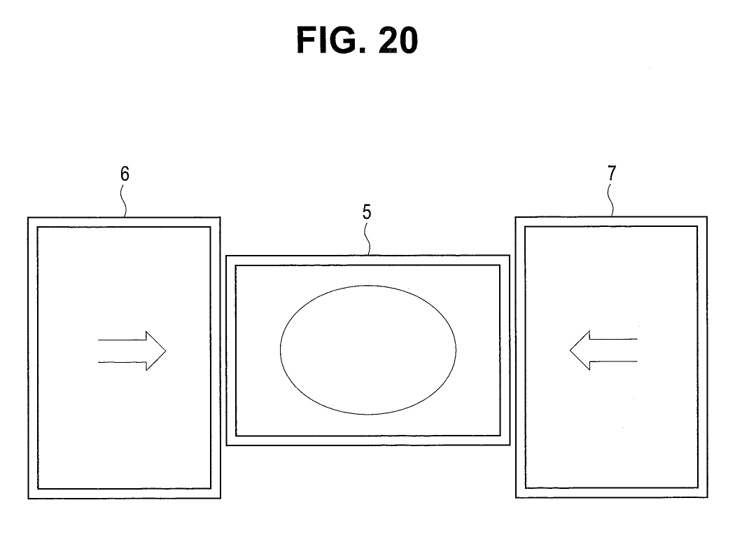

FIG. 18 is a plan view for describing a display example of an endoscopic image by three display devices, FIG. 19 is a plan view for describing one example of a display state of the three display devices, and FIG. 20 is a plan view illustrating one example of an installation state of the three display devices.

Incidentally, for the three display devices 5, 6 and 7 in the endoscope system 1, as illustrated in FIG. 18, the endoscopic image of a subject image made incident on the observation window 13 of the forward visual field is displayed at the display device 5 at the center, the endoscopic image of the subject image made incident on the observation window 16 on the left side which is one sideward visual field is displayed at the display device 6 arranged on the left, and the endoscopic image of the subject image made incident on the observation window 17 on the right side which is the other sideward visual field is displayed at the display device 7 arranged on the right.

At the time, at the left and right display devices 6 and 7, an advancing direction in which the insertion portion 10 of the endoscope 2 is inserted into the subject is upward indicated by an arrow in the figure.

That is, when the insertion portion 10 advances forward, the endoscopic images displayed at the left and right display devices 6 and 7 are displayed so as to move from an upper part to a lower part.

In contrast, in the endoscope system 1 of the present embodiment, the respective endoscopic images of the sideward visual field are rotated by 90 degrees by image processing by the control portion 32 of the processor 3 and displayed at the left and right display devices 6 and 7 such that the advancing direction of the insertion portion 10 of the endoscope 2 becomes the side of the center display device 5, as indicated by arrows in FIG. 19.

That is, for the advancing direction in which the insertion portion 10 of the endoscope 2 is inserted into the subject, the control portion 32 performs the image processing so that the right is the advancing direction as indicated by the arrow in the figure for the display device 6 on the left side, and the left is the advancing direction as indicated by the arrow in the figure for the display device 7 on the right side.

Therefore, when the insertion portion 10 advances forward, the endoscopic images displayed at the left and right display devices 6 and 7 are displayed so as to move from an inner part to an outer part. In other words, the endoscopic image of the display device 6 on the left side is displayed so as to move from the right to the left, and the endoscopic image of the display device 7 on the right side is displayed so as to move from the left to the right.

By such a configuration, in the endoscope system 1, in addition to the effects of the first embodiment or the second embodiment, movements of the endoscopic image when the insertion portion 10 is inserted can be displayed without an unnatural feeling.

### Modification

Note that the endoscope system 1 may be configured to arrange the left and right display devices 6 and 7 vertically so that a long side direction of an aspect ratio becomes vertical further, as illustrated in FIG. 20, and perform the image processing by the control portion 32 accordingly. Thus, the endoscopic image of the sideward visual field can be displayed in the wider range or enlarged and displayed.

### Fourth Embodiment

In the respective embodiments and the respective modifications described above, a mechanism that realizes a function of illuminating and observing the sideward direction is incorporated in the insertion portion 10 together with a mechanism that realizes a function of illuminating and observing the forward direction; however, the mechanism may be a separate body attachable and detachable to/from the insertion portion 10.

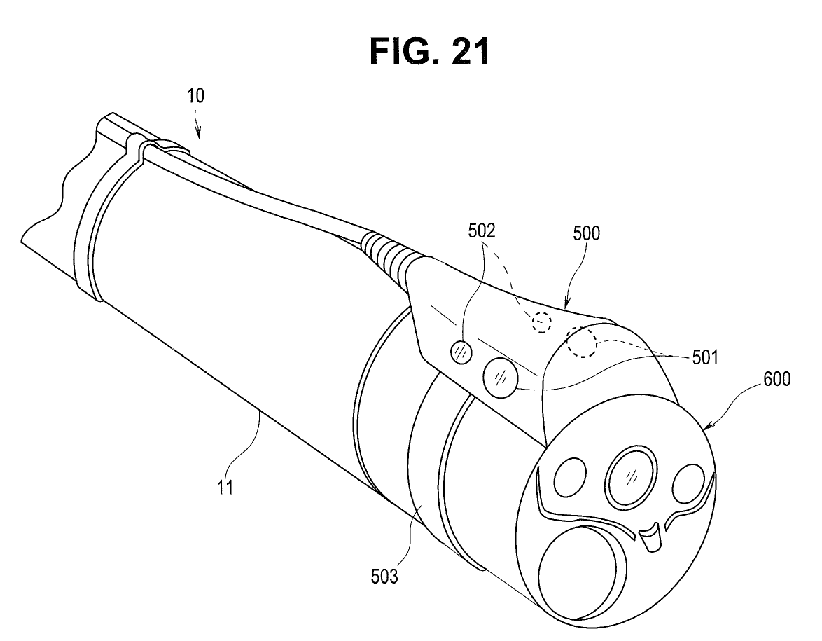

Note that FIG. 21 is a perspective view of the distal end portion 11 of the insertion portion 10 to which a side observation unit is attached relating to the fourth embodiment.

The distal end portion 11 of the insertion portion 10 includes a unit 600 for the forward visual field. A unit 500 for the sideward visual field is configured to be attached and detached to/from the unit 600 for the forward visual field by a clip portion 503.

The unit 500 for the sideward visual field includes two observation windows 501 for acquiring images in the lateral direction, and two illumination windows 502 configured to illuminate the lateral direction.

The processor 3 or the like lights and puts out the respective illumination windows 502 of the unit 500 for the sideward visual field according to a frame rate of the forward visual field, and observation images can be acquired and displayed as illustrated in the embodiments described above.

The invention described in the embodiments described above is not limited to the embodiments and the modifications and can be variously modified without departing from the scope in an implementation phase in addition. Further, the embodiments described above include the inventions in various stages, and various inventions can be extracted by appropriate combinations in a plurality of disclosed constituent elements.

For example, even when some constituent elements are omitted from the entire constituent elements indicated in the embodiments, in a case that a described problem can be solved and the described effects can be obtained, the configuration from which the constituent elements are omitted can be extracted as the invention.

What is claimed is:

1. An endoscope comprising:
an insertion portion to be inserted into an inside of a subject in a longitudinal axis direction;
a first image acquisition portion provided on a distal end face at a distal end of the insertion portion, and configured to acquire a first image from a first area including a forward direction of the insertion portion;
a second image acquisition portion provided on the distal end face at the distal end of the insertion portion, and configured to acquire a second image from a second area including a sideward direction of the insertion portion;
a first illumination light emission portion disposed on the distal end face at the distal end; and
at least two second illumination light emission portions lined up and provided so that the second image acquisition portion is interposed between the second illumination light emission portions at an angle to the longitudinal axis on a peripheral side face at the distal end,
wherein the second image acquisition portion is arranged at a position where a plane passing through the first image acquisition portion and the first illumination light emission portion passes through, and
the second illumination light emission portions are provided away from the plane, and an optical axes of illumination light radiated respectively from the two second illumination light emission portions has a predetermined angle to a normal line at a point where the optical axis of illumination light passes through on an outer peripheral portion of the insertion portion and has an angle in a direction separating from an image pickup optical axis of an image made incident on the second image acquisition portion.

2. The endoscope according to claim 1, wherein the second image acquisition portion is arranged at a position where a plane that passes through an optical axis of the first image acquisition portion and an optical axis of the first illumination light emission portion and is parallel to the longitudinal axis passes through.

3. The endoscope according to claim 1, wherein the two second illumination light emission portions are disposed on a straight line with the second image acquisition portion with the second image acquisition portion being interposed between the second illumination light emission portions.

4. The endoscope according to claim 3, wherein the two second illumination light emission portions are disposed at positions point-symmetrical to a center of the second image acquisition portion.

5. The endoscope according to claim 1, wherein the two second illumination light emission portions are lined up and disposed in a direction perpendicular to the longitudinal axis.

6. The endoscope according to claim 1, wherein the second image acquisition portion is positioned between arrangements of the two second illumination light emission portions.

7. The endoscope according to claim 1, wherein the sideward direction of the insertion portion is an area including a direction orthogonal to the longitudinal axis.

8. The endoscope according to claim 1, wherein the two second illumination light emission portions are arranged in a direction orthogonal to the direction connecting the first illumination light emission portion in a pair.

9. The endoscope according to claim 1, comprising:
a first image pickup portion configured to photoelectrically convert the first image; and
a second image pickup portion different from the first image pickup portion, configured to photoelectrically convert the second image.

10. An endoscope comprising:
an insertion portion to be inserted into an inside of a subject in a longitudinal axis direction;
a first image acquisition portion provided on a distal end face of the insertion portion, and configured to acquire a first image from a first area including a forward direction of the insertion portion;
a second image acquisition portion provided on a peripheral side face of the insertion portion, and configured to acquire a second image from a second area including a sideward direction of the insertion portion;
a pair of first illumination light emission portions arranged with the first image acquisition portion being interposed between the pair of first illumination light emission portions along a line orthogonal to an optical axis of the first image acquisition portion, on the distal end face of the insertion portion; and
two roughly L-shaped second illumination light emission portions disposed so as to surround the second image acquisition portion, on the peripheral side face of the insertion portion.

11. An endoscope system comprising:
the endoscope according to claim 1;
an image processing portion configured to arrange the second image from the second image acquisition portion so as to be adjacent to the first image from the first image acquisition portion; and
an image output portion configured to generate a display signal for causing a display portion to perform display, based on a signal relating to the first image and a signal relating to the second image from the image processing portion.
